(19) United States
(12) Patent Application Publication
(10) Pub. No.: US 2024/0136010 A1
(43) Pub. Date: Apr. 25, 2024

(54) METHOD FOR CREATING CELL MATHEMATICAL MODEL, CELL MATHEMATICAL MODEL CREATION PROGRAM, CELL MATHEMATICAL MODEL CREATION APPARATUS, METHOD FOR DETERMINING CELL MATHEMATICAL MODEL, CELL MATHEMATICAL MODEL DETERMINATION PROGRAM, AND CELL MATHEMATICAL MODEL DETERMINATION APPARATUS

(71) Applicant: FUJIFILM Corporation, Tokyo (JP)

(72) Inventors: Takahiro TERAO, Ashigarakami-gun (JP); Masaya NAGASE, Ashigarakami-gun (JP)

(73) Assignee: FUJIFILM Corporation, Tokyo (JP)

(21) Appl. No.: 18/397,262

(22) Filed: Dec. 27, 2023

Related U.S. Application Data
(63) Continuation of application No. PCT/JP2022/019690, filed on May 9, 2022.

(30) Foreign Application Priority Data
Jun. 28, 2021 (JP) 2021-107842

(51) Int. Cl. G16B 5/00; G16B 40/00

(52) U.S. Cl. CPC G16B 5/00; G16B 40/00

(57) ABSTRACT

Provided are a method for creating a cell mathematical model capable of evaluating characteristics of cells from experimental data, a program, and a creation apparatus. Also provided are a method for determining the created cell mathematical model, a program, and a determination apparatus. The method for creating a mathematical model receives culture data of the cells, extracts feature values of cell activity from the culture data, creates a cell mathematical model from the feature values, and outputs the cell mathematical model. The method for determining the created cell mathematical model determines that estimated culture data calculated using the cell mathematical model reflects the culture data.

### CROSS-REFERENCE TO RELATED APPLICATIONS

The present application is a Continuation of PCT International Application No. PCT/JP2022/019690 filed on May 9, 2022 claiming priority under 35 U.S.C. § 119(a) to Japanese Patent Application No. 2021-107842 filed on Jun. 29, 2021. Each of the above applications is hereby expressly incorporated by reference, in its entirety, into the present application.

### BACKGROUND OF THE INVENTION

### 1. Field of the Invention

The present invention relates to a method for creating a cell mathematical model, a cell mathematical model creation program, a cell mathematical model creation apparatus, a method for determining a cell mathematical model, a cell mathematical model determination program, and a cell mathematical model determination apparatus, and more specifically to a method for creating a cell mathematical model, a cell mathematical model creation program, a cell mathematical model creation apparatus, a method for determining a cell mathematical model, a cell mathematical model determination program, and a cell mathematical model determination apparatus for use in searching for culture conditions.

### 2. Description of the Related Art

To grasp the biological functions and abilities of cultured cells, it is essential to understand the characteristics of the cells. In particular, in the production of biopharmaceuticals, it is important to grasp the characteristics of cultured cells to determine the suitability of production cells and culture conditions. Also in the elucidation of pathological mechanisms, it is important to know the relationship between cell characteristics and environmental factors. As a method for implementing them, techniques have been developed that focus on intracellular metabolism and that determine biological behaviors and characteristics such as the amount of metabolic reaction and growth only from the structure of a metabolic reaction circuit.

Metabolic flux balance analysis (FBA) uses only the structure of a metabolic reaction to analyze the behavior range and features of a target metabolic circuit on the basis of basic constraint conditions such as the law of conservation of mass even when constants related to metabolism are not fully measurable. For example, Shilling, C. and Palsson, B., Proc. Natl. Acad. Sci. USA, vol. 95, pp. 4193-4198, 1988 describes that, first, a metabolic reaction is described as a series of linear equations, a vector space of solutions to the simultaneous equations is defined, the vector space is converted into a biochemically meaningful basis, and, finally, linear programming is used to identify a metabolic state that maximizes a given objective function.

Further, H. Hefzi, N. E. Lewis, Cell Systems 3, 434-443, 2016 describes, as a method for reflecting characteristics of cells in a mathematical model for FBA (FBA model), a method using genome information, gene expression level information, and protein expression level information. This is a method for directly acquiring and determining the presence of a gene indicating the presence or absence of a metabolic reaction in an FBA model.

### SUMMARY OF THE INVENTION

However, the methods described in Shilling, C. and Palsson, B., Proc. Natl. Acad. Sci. USA, 95, 4193-4198, 1988 and H. Hefzi, N. E. Lewis, Cell Systems 3, 434-443, 2016 are to create a model obtained by reflecting, in an FBA mathematical model, characteristics of cells from gene expression data including cell characteristic information. For this reason, gene expression data needs to be acquired, and the model may be difficult to create.

The present invention has been made in view of such circumstances, and provides a method for creating a cell mathematical model reflecting cellular individuality from experimental data, a cell mathematical model creation program, and a cell mathematical model creation apparatus. The present invention further provides a method for determining the created cell mathematical model, a cell mathematical model determination program, and a cell mathematical model determination apparatus.

To achieve the objects of the present invention, a method for creating a cell mathematical model according to the present invention includes receiving culture data of cells, extracting feature values of cell activity from the culture data, creating a cell mathematical model from the feature values, and outputting the cell mathematical model.

According to an aspect of the present invention, preferably, the culture data includes the number of cells, an amount of a cell-secreted substance, an amount of a cell-produced substance, an amount of a cell metabolite, a culture medium component amount, temporal change data of the number of cells, temporal change data of the amount of the cell-secreted substance, temporal change data of the amount of the cell-produced substance, temporal change data of the amount of the cell metabolite, and temporal change data of the culture medium component amount.

According to an aspect of the present invention, preferably, the method for creating a cell mathematical model further includes separating the feature values of the cell activity are separated into an input factor into the cells and an output factor from the cells, and in the creating of a cell mathematical model, a cell mathematical model satisfying the input factor and the output factor at any time is created.

According to an aspect of the present invention, preferably, the feature values of the cell activity are a concentration of a culture medium component, a consumption amount or consumption rate of the culture medium component, a generation amount or generation rate of a cell-secreted substance, a production amount or production rate of a cell-produced substance, and a generation amount or generation rate of a cell metabolite.

According to an aspect of the present invention, preferably, the input factor is the concentration of the culture medium component, and the output factor includes the consumption amount or consumption rate of the culture medium component and at least one of the generation amount or generation rate of the cell-secreted substance, the production amount or production rate of the cell-produced substance, or the generation amount or generation rate of the cell metabolite.

According to an aspect of the present invention, preferably, for the concentration of the culture medium component, a theoretical upper limit amount absorbable by the cells, or a consumption rate is calculated.

According to an aspect of the present invention, preferably, the theoretical upper limit amount absorbable by the cells, or the consumption rate is calculated by using Michaelis-Menten equation or Fick's law.

According to an aspect of the present invention, preferably, in the creating of a cell mathematical model, a reference cell mathematical model is randomly modified to generate a cell mathematical model.

According to an aspect of the present invention, preferably, the reference cell mathematical model is randomly modified by using a genetic algorithm.

According to an aspect of the present invention, preferably, the cells are eukaryotic cells or prokaryotic cells.

According to an aspect of the present invention, preferably, the eukaryotic cells are animal, plant, or insect derived cell lines, primary cultures, or fungi.

According to an aspect of the present invention, preferably, the prokaryotic cells are bacteria including Escherichia coli, Bacillus subtilis, cyanobacteria, or actinomycetes, and archaebacteria including methanogens, hyperhalophiles, or hyperthermophiles.

To achieve the objects of the present invention, a cell mathematical model creation program according to the present invention causes a computer to execute the method for creating a cell mathematical model described above.

To achieve the objects of the present invention, a cell mathematical model creation apparatus according to the present invention includes a processor configured to receive culture data; extract feature values of cell activity from the culture data; create a cell mathematical model from the feature values; and output the cell mathematical model.

To achieve the objects of the present invention, a method for determining a cell mathematical model according to the present invention is a method for determining the cell mathematical model created by the method for creating a cell mathematical model described above, including determining that estimated culture data calculated using the cell mathematical model reflects the culture data.

According to an aspect of the present invention, preferably, in the determining that the estimated culture data reflects the culture data, it is checked that the cell mathematical model is established between an input factor and an output factor for the cells at a plurality of timings in time-series data obtained by acquiring the culture data.

According to an aspect of the present invention, preferably, in the determining that the estimated culture data reflects the culture data, it is checked that the cell mathematical model is established between an initial input factor into the cells in the culture data and an output factor calculated continuously over time.

According to an aspect of the present invention, preferably, in the determining that the estimated culture data reflects the culture data, a determination is performed using a sum of absolute values of differences between the culture data and the estimated culture data in respective elapsed amounts of time.

According to an aspect of the present invention, preferably, in the determining that the estimated culture data reflects the culture data, a determination is performed on the basis of a difference between a calculation value obtained by a cell simulator in which the cell mathematical model is incorporated and the culture data.

To achieve the objects of the present invention, a cell mathematical model determination program according to the present invention causes a computer to execute the method for determining a cell mathematical model described above.

To achieve the objects of the present invention, a cell mathematical model determination apparatus according to the present invention includes a processor configured to receive culture data; extract feature values of cell activity from the culture data; create a cell mathematical model from the feature values; output the cell mathematical model; and determine that estimated culture data calculated using the cell mathematical model reflects the culture data.

According to the present invention, it is possible to create a cell mathematical model reflecting cellular individuality from experimental data. It is also possible to determine the created cell mathematical model.

### BRIEF DESCRIPTION OF THE DRAWINGS

FIG. 1 is a block diagram illustrating a configuration of a cell mathematical model creation apparatus;

FIG. 2 is a block diagram illustrating a configuration of a processing unit;

FIG. 3 is a flowchart illustrating a method for creating a cell mathematical model;

FIG. 4 is a diagram illustrating a feature value extraction step;

FIG. 5 is a schematic diagram illustrating cellular metabolic pathways;

FIG. 6 is a diagram illustrating an overview of the Michaelis-Menten equation;

FIG. 7 is a flowchart illustrating a mathematical model creation step;

FIG. 8 is a diagram illustrating the mathematical model creation step;

FIG. 9 is a block diagram illustrating a configuration of a processing unit of a cell mathematical model determination apparatus;

FIG. 10 is a flowchart illustrating a method for determining a cell mathematical model;

FIG. 11 is a diagram illustrating a mathematical model determination step;

FIG. 12 is a diagram illustrating an overview of a simulation using a cell simulator; and

FIG. 13 depicts graphs illustrating the results of an example.

### DESCRIPTION OF THE PREFERRED EMBODIMENTS

A method for creating a cell mathematical model, a cell mathematical model creation program, a cell mathematical model creation apparatus, a method for determining a cell mathematical model, a cell mathematical model determination program, and a cell mathematical model determination apparatus according to embodiments of the present invention will be described hereinafter with reference to the accompanying drawings.

### Cell Mathematical Model Creation Apparatus

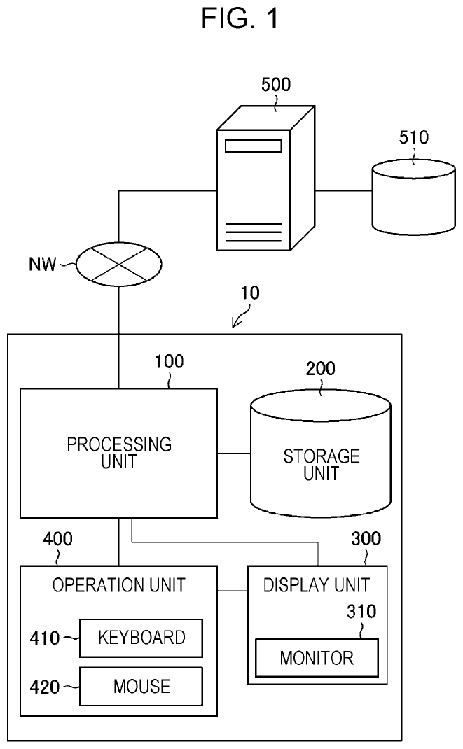

FIG. 1 is a block diagram illustrating a configuration of a cell mathematical model creation apparatus 10 (hereinafter also simply referred to as “model creation apparatus”). The model creation apparatus 10 is an apparatus that creates a cell mathematical model on the basis of input culture data of cells, and can be implemented using a computer. As illustrated in FIG. 1, the model creation apparatus 10 includes a processing unit 100, a storage unit 200, a display unit 300, and an operation unit 400, which are connected to each other to transmit and receive necessary information. These components can be installed in various ways. The components may be installed in one location (such as in one housing or one room) or may be installed in separate locations and connected to each other via a network. The model creation apparatus 10 can be connected to an external server 500 and external data 510 via a network NW such as the Internet to acquire information such as culture data and the created cell mathematical model, as necessary.

### Configuration of Processing Unit

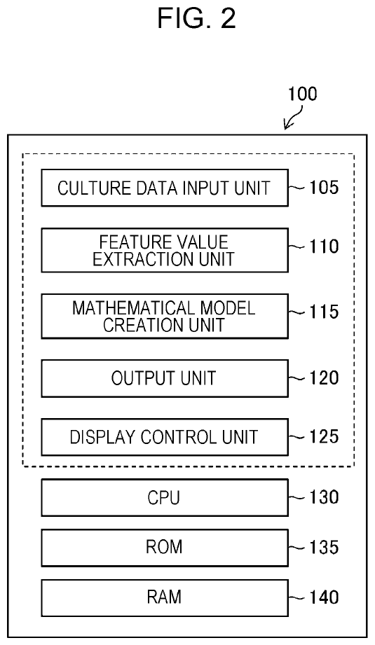

FIG. 2 is a diagram illustrating a configuration of the processing unit 100. The processing unit 100 includes a culture data input unit 105, a feature value extraction unit 110, a mathematical model creation unit 115, an output unit 120, a display control unit 125, a CPU 130 (CPU: Central Processing Unit), a ROM 135 (ROM: Read Only Memory), and a RAM 140 (RAM: Random Access Memory).

The culture data input unit 105 receives input culture data. The feature value extraction unit 110 extracts feature values of cell activity on the basis of the culture data input to the culture data input unit 105. The mathematical model creation unit 115 creates a cell mathematical model from the feature values of the cell activity extracted by the feature value extraction unit 110. The output unit 120 outputs the cell mathematical model created by the mathematical model creation unit 115. The display control unit 125 controls display of the acquired information and processing results on a monitor 310. A method for creating a cell mathematical model by using these functions of the processing unit 100 will be described in detail. The processing by these functions is performed under the control of the CPU 130.

The functions of the components of the processing unit 100 described above can be implemented using various processors. The various processors include, for example, a CPU, which is a general-purpose processor that executes software (program) to implement various functions. The various processors described above also include a programmable logic device (PLD) that is a processor whose circuit configuration can be changed after manufacture, such as an FPGA (Field Programmable Gate Array). The various processors described above further include a dedicated electric circuit that is a processor having a circuit configuration designed specifically for executing specific processing, such as an ASIC (Application Specific Integrated Circuit).

The functions of the components may be implemented by a single processor or a combination of a plurality of processors. Alternatively, a plurality of functions may be implemented by a single processor. Examples of configuring a plurality of functions by a single processor include, first, a form in which, as typified by a computer such as a client or server computer, the single processor is configured by a combination of one or more CPUs and software and the processor is implemented as a plurality of functions. The examples include, second, a form in which, as typified by a system on chip (SoC) or the like, a processor is used in which the functions of the entire system are implemented by a single IC (Integrated Circuit) chip. As described above, the various functions are configured using one or more of the various processors described above as a hardware structure. More specifically, the hardware structure of the various processors is an electric circuit (circuitry) including a combination of circuit elements such as semiconductor elements.

When the processor or electric circuit described above is to execute software (program), the processor (computer) readable code of the software to be executed is stored in a non-transitory recording medium such as the ROM 135 (see FIG. 2), and the processor refers to the software. The software to be stored in the non-transitory recording medium includes a program for executing the method for creating a cell mathematical model according to the present embodiment. The code may be recorded on a non-transitory recording medium such as various magneto-optical recording devices or semiconductor memories, instead of the ROM 135. In the processing using software, for example, the RAM 140 is used as a temporary storage area. For example, data stored in an EEPROM (Electrically Erasable and Programmable Read Only Memory) (not illustrated) can be referred to.

### Configuration of Storage Unit

The storage unit 200 is constituted by a non-transitory recording medium such as a DVD (Digital Versatile Disk), a hard disk, or various semiconductor memories, and a control unit therefor. The storage unit 200 stores the culture data input to the culture data input unit 105, the feature values of the cell activity extracted by the feature value extraction unit 110, and the cell mathematical model created by the mathematical model creation unit 115.

### Configuration of Display Unit and Operation Unit

The display unit 300 includes the monitor 310 (display device) and is capable of displaying input information, information stored in the storage unit 200, a result of processing performed by the processing unit 100, and so on. The operation unit 400 includes a keyboard 410 and a mouse 420 as an input device and/or a pointing device. The user can use these devices and the screen of the monitor 310 to perform operations necessary to execute the method for creating a cell mathematical model according to the present embodiment. The operations that the user can perform include input of culture data, for example.

### Process in Cell Mathematical Model Creation Apparatus

The model creation apparatus 10 described above can create a cell mathematical model in accordance with an instruction given by the user through the operation unit 400.

### Method for Creating Cell Mathematical Model

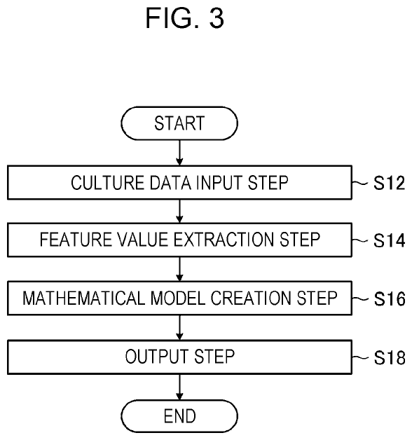

FIG. 3 is a flowchart illustrating the method for creating a cell mathematical model according to the present embodiment. The method for creating a cell mathematical model according to the present embodiment has a culture data input step (step S12) of receiving culture data of cells, a feature value extraction step (step S14) of extracting feature values of cell activity from the culture data input in the culture data input step, a mathematical model creation step (step S16) of creating a cell mathematical model from the feature values of the cell activity extracted in the feature value extraction step, and an output step (step S18) of outputting the mathematical model created in the mathematical model creation step.

The steps will be described hereinafter.

### Culture Data Input Step (Step S12)

The culture data input unit 105 of the model creation apparatus 10 performs a culture data input step (step S12). The culture data input step is a step of receiving culture data of cells. The culture data is input by the user.

The culture data includes the number of cells, the amount of a cell-secreted substance, the amount of a cell-produced substance, the amount of a cell metabolite, the culture medium component amount, and temporal change data thereof. As used herein, the cell-secreted substance refers to a substance that is not a substance of interest among substances generated by the cells and produced extracellularly. Examples of the cell-secreted substance include ammonia and by-products. The cell-produced substance refers to a substance of interest among substances generated by the cells and produced extracellularly. Examples of the cell-produced substance include antibodies. The cell metabolite refers to a substance present in the cells among substances generated by the cells. As the culture data, culture data obtained by actually performing culture can be used.

### Feature Value Extraction Step (Step S14)

The feature value extraction unit 110 of the model creation apparatus 10 performs a feature value extraction step (step S14). The feature value extraction step extracts feature values of cell activity from the culture data input in the culture data input step.

The feature values of the cell activity are the concentration of a culture medium component, the consumption amount or consumption rate of the culture medium component, the generation amount or generation rate of the cell-secreted substance, the production amount or production rate of the cell-produced substance, and the generation amount or generation rate of the cell metabolite. In the feature value extraction step, the feature values of the cell activity described above are extracted from the culture data input in the culture data input step.

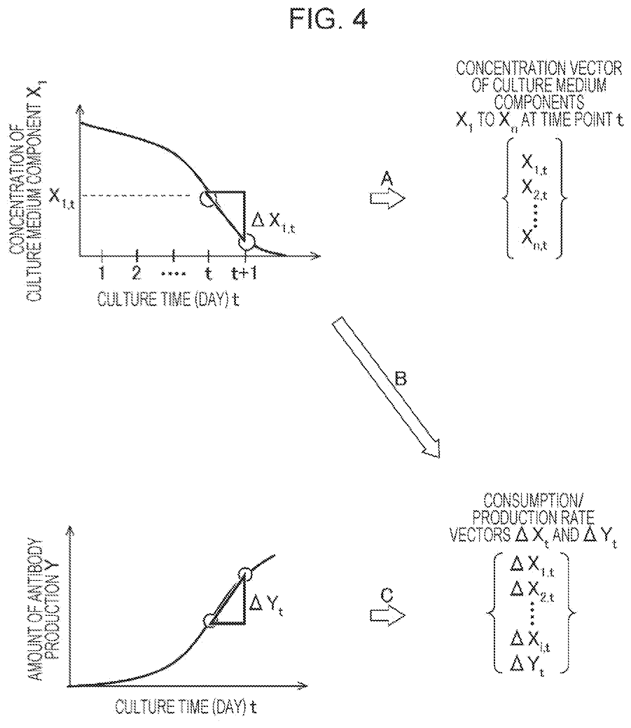

FIG. 4 is a diagram illustrating the feature value extraction step. In FIG. 4, time-series culture data of culture medium component concentrations (for a component X₁ as an example) and time-series culture data of the amounts of antibody production (Y) are illustrated. In the feature value extraction step, for example, concentration vectors of culture medium components X₁ to Xₙ at a point in culture time t are extracted from the time-series culture data of the culture medium component concentrations illustrated in FIG. 4, as indicated by an arrow A. Further, as indicated by an arrow B, consumption rates ΔX_{1,t} to ΔX_{n,t} of the culture medium components X₁ to Xₙ are extracted. The consumption rate ΔX_{1,t} of the culture medium component X₁ is determined by dividing a consumption amount ΔX₁ of the culture medium component X₁ from time t to time (t+1) by the culture time to determine the consumption rate ΔX_{1,t} at the time t. The consumption rates ΔX_{2,t} to ΔX_{n,t} of the other culture medium components can also be determined in a similar manner. Likewise, as indicated by an arrow C, a production rate ΔYₜ of an antibody Y is extracted from the time-series culture data of the amounts of antibody production. The production rate ΔYₜ of the antibody Y is determined by dividing the amount of increase ΔY of the antibody Y from the time t to the time (t+1) by the culture time to determine the production rate ΔYₜ at the time t.

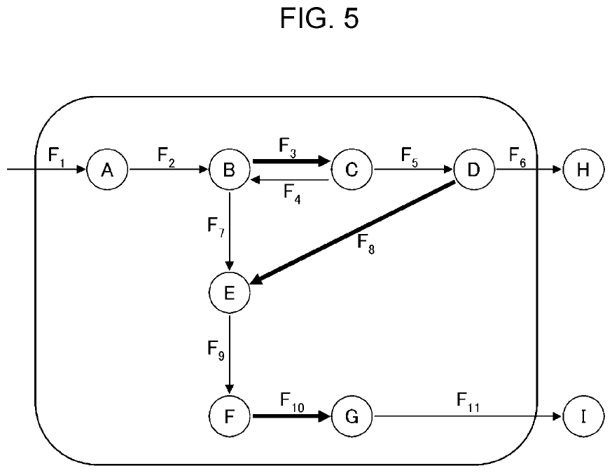

For the concentration of a culture medium component, preferably, a theoretical upper limit amount of the culture medium component absorbable by cells or a consumption rate at which the cells consume the culture medium component is calculated, and the concentration vector of the culture medium component is extracted. FIG. 5 is a schematic diagram illustrating an example of cellular metabolic pathways. In FIG. 5, substances generated by metabolism are represented by A, B, C, etc. F₁, F₂, F₃, etc. represent functions indicating the respective changes in the concentrations of the substances over time. For example, F₁ represents a flux at which the substance A is taken in, and F₂ represents a flux at which the substance B is made through metabolism.

In the example illustrated in FIG. 5, the conversion of the substance A into the substance B is performed in equal amount in accordance with the law of conservation of mass. Likewise, the amount of the substance B is equal to the total amount of conversion into the substance C and the substance E. As described above, the amount of a substance to be converted is determined by the amounts of a substrate and a nutrient to be initially taken in by cells, and so on. Another parameter that limits the conversion of a substance is the reaction rate. For example, as described above, the conversion of the substance A into the substance B is performed in equal amount. However, in a certain unit time, the conversion into the substance B has an upper limit due to the limited rate of reaction, and not all the substance A may be converted into the substance B. Accordingly, when the concentration of a culture medium component is high, a difference may occur between the actual concentration of the culture medium component and the concentration of a culture medium component that can be consumed by culture. For this reason, if a cell mathematical model is created by using the actual concentration of the culture medium component, it is feared that the creation of the cell mathematical model to be created will not be based on the cell characteristics. Accordingly, the theoretical upper limit amount of a culture medium that can be absorbed by cells or the consumption rate of the culture medium is calculated and used as a feature value of cell activity. This makes it possible to create a cell mathematical model that is more based on the cell characteristics.

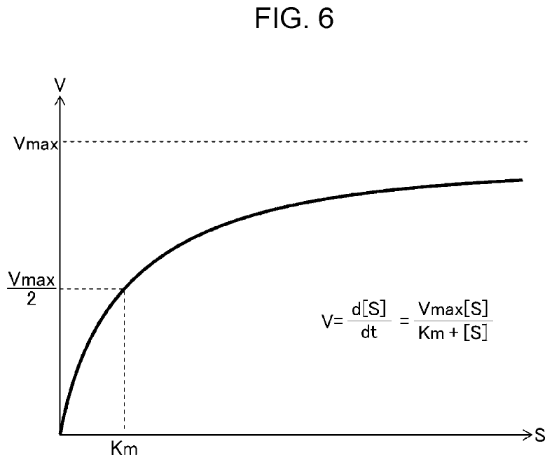

The theoretical upper limit amount that can be absorbed by cells or the consumption rate of the culture medium can be calculated by using, for example, the Michaelis-Menten equation. FIG. 6 illustrates an overview of the Michaelis-Menten equation. The Michaelis-Menten equation is an equation related to a reaction rate V of an enzyme. When a substrate concentration S is low, the reaction rate V is proportional to the substrate concentration S. When the substrate concentration S is high, the reaction rate V converges to a maximum rate Vmax regardless of the substrate concentration S. Other mathematical models that can be used include Fick's law. Fick's law is an expression used to determine a flux that is the amount of substance passing through a unit area per unit time, and the flux is proportional to a diffusion coefficient D and a substrate concentration gradient on both sides of a membrane. The Michaelis-Menten equation or Fick's law is used to calculate the culture medium concentration. This makes it possible to create a cell mathematical model that is more based on the cell characteristics.

### Mathematical Model Creation Step (Step S16)

The mathematical model creation unit 115 of the model creation apparatus 10 performs a mathematical model creation step (step S16). The mathematical model creation step is a step of generating a cell mathematical model from the feature values of the cell activity extracted in the feature value extraction step.

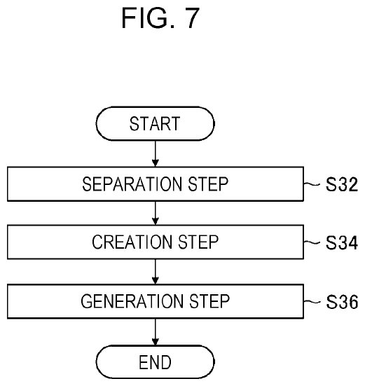

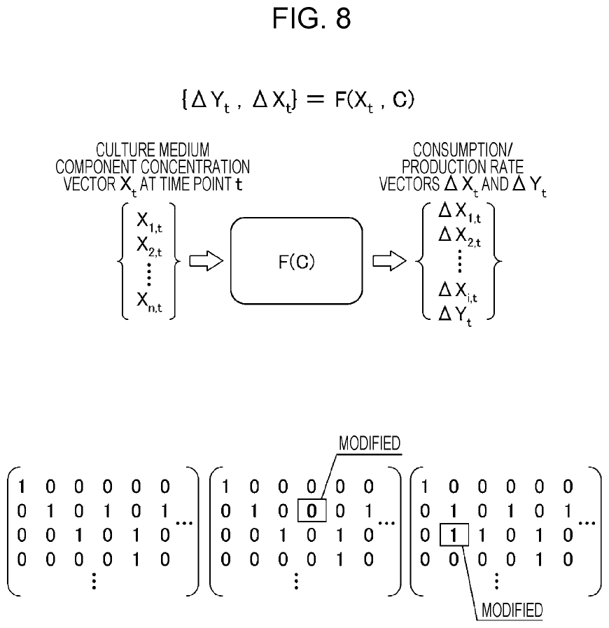

FIG. 7 is a flowchart illustrating the mathematical model creation step. FIG. 8 is a diagram illustrating the mathematical model creation step.

The cell mathematical model creation step separates the feature values of the cell activity extracted in the feature value extraction step into input factors for the cells and output factors from the cells (separation step: step S32). In the separation step, the concentration of a culture medium component is selected as an input factor. Further, an output factor is selected so as to include at least one of the consumption amount or consumption rate of the culture medium component, the generation amount or generation rate of a cell-secreted substance, the production amount or production rate of a cell-produced substance, or the generation amount or generation rate of a cell metabolite.

Then, as illustrated in FIG. 8, F(C) satisfying the input factor and the output factor is searched for to create (search for) a cell mathematical model (creation step: step S34). The model is created by using, here, the concentrations of the culture medium components X₁ to Xₙ at the time point t as input factors and using, as output factors, the consumption rates ΔX_{1,t} to ΔX_{n,t} of the culture medium components X₁ to Xₙ at the time point t and the production rate ΔYₜ of the antibody Y at the time point t. The creation (search) of a cell mathematical model is performed by creating a cell mathematical model that reproduces feature values of cell activity, for which the input factors and the output factors are satisfied, at any time points ta, t₂, t₃, t₄, etc.

Further, the cell mathematical model is based on, as a reference, a cell mathematical model that reproduces the feature values of the cell activity, and the mathematical model creation unit 115 can modify this reference cell mathematical model to generate a plurality of cell mathematical models (generation step: step S36). As illustrated in FIG. 8, the modification of the cell mathematical model can be performed by randomly modifying a reference cell mathematical model. For example, as illustrated in FIG. 8, in a cell mathematical model on the center, the numerical value in the second row and the fourth column is modified from the corresponding value of a reference cell mathematical model (on the left). In a cell mathematical model on the right, the numerical value in the third row and the second column is modified from the corresponding value of the reference cell mathematical model. Further, in random modification, the modification can be performed by using a genetic algorithm. The genetic algorithm is a method for creation through repeated operations such as mutation and crossover. Mutation in the genetic algorithm is an operation of changing a portion of the cell mathematical model. Crossover is a method for selecting one crossover point between two cell mathematical models and swapping portions after the position of the crossover point to create another cell mathematical model.

Through the mathematical model creation step (step S16), a cell mathematical model that reproduces feature values of cell activity can be created. This mathematical model is used as a reference, and a portion of the cell mathematical model is modified to create a cell mathematical model. This makes it possible to easily search for another cell mathematical model that can reproduce culture data even if culture data estimated by using a cell mathematical model that reproduces feature values of cell activity is not a reproduction of the actual culture data.

### Output Step (Step S18)

The output unit 120 of the model creation apparatus 10 performs an output step (step S18). The output step is a step of outputting the cell mathematical model created in the mathematical model creation step (step S16). The cell mathematical model output from the output unit 120 is displayed on the monitor 310.

As a result output in the output step, a plurality of cell mathematical models created in the mathematical model creation step can be output.

According to the method for creating a cell mathematical model, the cell mathematical model creation program, and the cell mathematical model creation apparatus according to the embodiment of the present invention, it is possible to create a cell mathematical model by using culture data obtained by actual culture. This makes it possible to create a cell mathematical model from the culture data without requiring gene-level information. In other words, according to the method for creating a cell mathematical model, the cell mathematical model creation program, and the cell mathematical model creation apparatus according to the embodiment of the present invention, it is possible to evaluate cell characteristics reflecting cellular individuality by using culture data that can be easily acquired without obtaining measurement data that requires a large amount of cost, such as gene expression data.

### Cell Mathematical Model Determination Apparatus

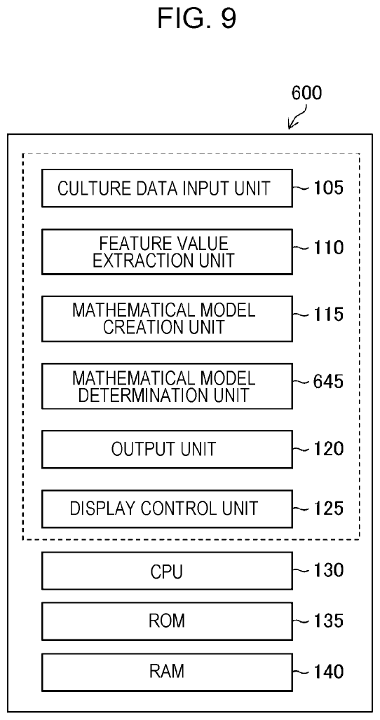

Next, a cell mathematical model determination apparatus will be described. The cell mathematical model determination apparatus (hereinafter also simply referred to as “model determination apparatus”) is an apparatus that determines a cell mathematical model created on the basis of input culture data of cells, and is implemented using a computer. Like the model creation apparatus 10 illustrated in FIG. 1, the model determination apparatus includes a processing unit 600 (see FIG. 9), a storage unit 200, a display unit 300, and an operation unit 400, which are connected to each other to transmit and receive necessary information. The components other than the processing unit 600 have configurations and functions similar to those of the model creation apparatus 10, and thus the description thereof will be omitted.

### Configuration of Processing Unit

FIG. 9 is a diagram illustrating a configuration of the processing unit 600. The processing unit 600 includes a culture data input unit 105, a feature value extraction unit 110, a mathematical model creation unit 115, a mathematical model determination unit 645, an output unit 120, a display control unit 125, a CPU 130, a ROM 135, and a RAM 140. The culture data input unit 105, the feature value extraction unit 110, the mathematical model creation unit 115, the display control unit 125, the CPU 130, the ROM 135, and the RAM 140 have configurations and functions similar to those of the components disposed in the processing unit 100 of the model creation apparatus 10, and thus the description thereof will be omitted. The mathematical model determination unit 645 determines whether the cell mathematical model created by the mathematical model creation unit 115 is a cell mathematical model that reflects the culture characteristics of the cells. The output unit 120 outputs the cell mathematical model having the lowest score determined by the mathematical model determination unit 645 or all the cell mathematical models having scores within a reference value. A cell mathematical model determination method using these functions of the processing unit 600 will be described in detail. The processing by these functions is performed under the control of the CPU 130.

The functions of the components of the processing unit 600 described above can be implemented using various processors. When a processor or electric circuit is to execute software (program), the processor (computer) readable code of the software to be executed is stored in a non-transitory recording medium such as the ROM 135, and the processor refers to the software. The software to be stored in the non-transitory recording medium includes a program for executing the method for determining a cell mathematical model according to the present embodiment.

### Method for Determining Cell Mathematical Model

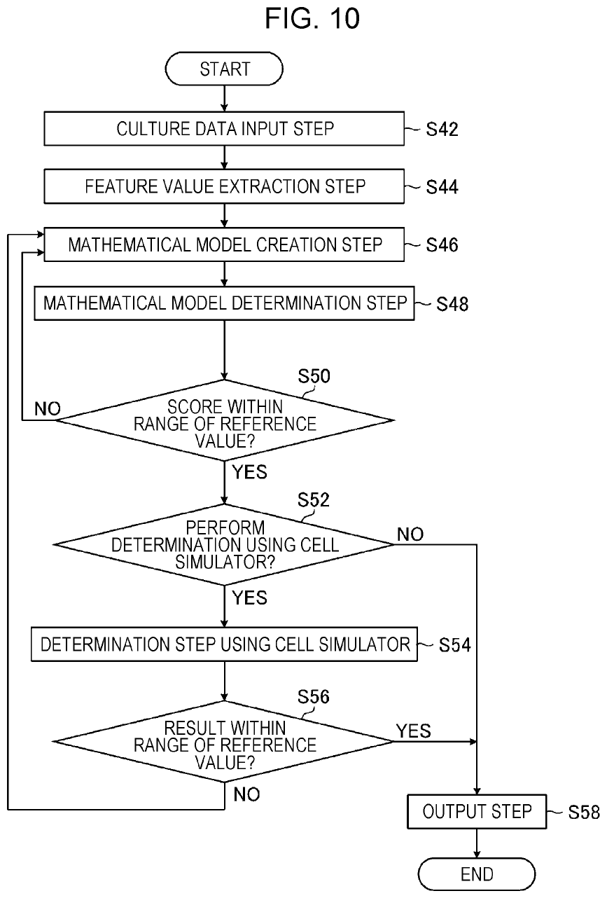

FIG. 10 is a flowchart illustrating the method for determining a cell mathematical model according to the present embodiment. The method for determining a cell mathematical model according to the present embodiment has a culture data input step (step S42) of receiving culture data of cells, a feature value extraction step (step S44) of extracting feature values of cell activity from the culture data input in the culture data input step, a mathematical model creation step (step S46) of creating a cell mathematical model from the feature values of the cell activity extracted in the feature value extraction step, and a mathematical model determination step (step S48) of determining that estimated culture data calculated by using the cell mathematical model created in the mathematical model creation step reflects the culture data input in the culture data input step. The method further has an output step (step S58) of outputting the cell mathematical model created in the mathematical model creation step and a result determined in the mathematical model determination step.

### Culture Data Input Step (Step S42) to Mathematical Model Creation Step (Step S46)

The culture data input step (step S42), the feature value extraction step (step S44), and the mathematical model creation step (step S46) can be performed in a way similar to that of the method for creating a cell mathematical model illustrated in FIG. 3, and thus the description thereof will be omitted.

### Mathematical Model Determination Step (Step S48)

The mathematical model determination unit 645 of the model determination apparatus performs a mathematical model determination step (step S48). The mathematical model determination step is a step of determining the cell mathematical model created in the mathematical model creation step, and compares estimated culture data calculated by using the created cell mathematical model with the culture data input to the culture data input unit 105 to determine the created cell mathematical model.

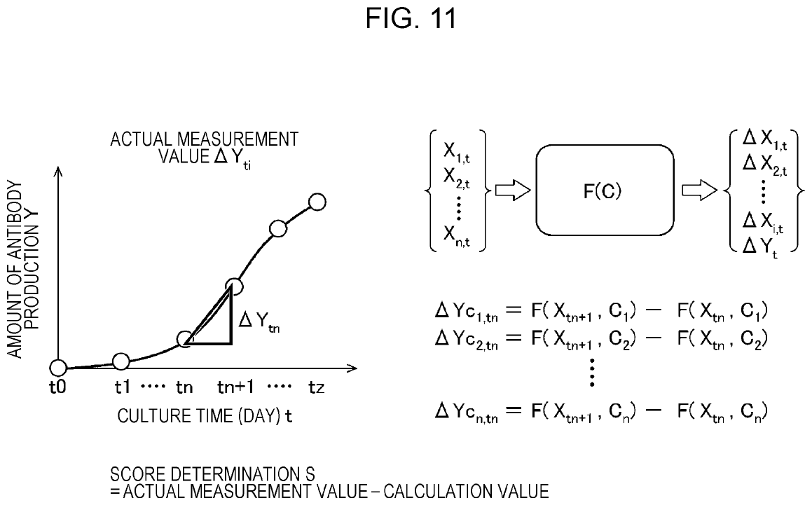

FIG. 11 is a diagram illustrating the mathematical model determination step. FIG. 11 is a diagram illustrating an example of determining a cell mathematical model on the basis of culture data for antibody production. The mathematical model determination step makes a determination by assigning a score to the created cell mathematical model. For example, a cell mathematical model C₁ is used. As illustrated in FIG. 11, the production amount of the antibody Y at a time point tn can be determined by ΔY_{C1,tn}=F(Xₜₙ₊₁, C₁)−F(Xₜₙ, Cₜ). An actual measurement value ΔYₜₙ can be determined from the actual measurement value of the culture data by using ΔYₜₙ=(Yₜₙ₊₁−Yₜₙ)/(tn+1−tn). A score Sₜₙ is determined by “actual measurement value (ΔYₜₙ)−calculation value (ΔY_{C1,tn}) using the cell mathematical model”. This calculation is performed for each of time points t₀, t₁, t₂, t₃, tn, . . . , and tz in an elapsed amount of time to determine scores Sₜ₀, Sₜ₁, Sₜ₂, Sₜ₃, Sₜₙ, . . . , and S_{tz} at the respective time points by using the cell mathematical model C₁. The total Sₜₒₜₐₗ of the absolute values of the scores at the respective time points (Sₜₒₜₐₗ=Sₜ₀+Sₜ₁+St₂+Sₜ₃+ . . . +Sₜₙ+ . . . +S_{tz}) is the score of the cell mathematical model C₁.

In the mathematical model creation step, a plurality of (n) cell mathematical models are created in the generation step, and the score Sₜₒₜₐₗ is also detected for these cell mathematical models (C₂ to Cₙ) by using a similar method.

The scores Sₜₒₜₐₗ of the cell mathematical models (C₁ to Cₙ) can be compared, and the cell mathematical model having the lowest score can be selected as a model reflecting the cell characteristics of the cells used for culture. In other words, the mathematical model determination unit 645 selects the cell mathematical model having the smallest difference between the actual measurement value and the calculation value obtained by using the cell mathematical model as a model reflecting the cell characteristics of the cells used for culture. Further, the mathematical model determination unit 645 can determine a reference value of the scores and select a cell mathematical model whose score Sₜₒₜₐₗ falls within the range of the reference value.

In the foregoing description, the cell mathematical model is determined by using the total Sₜₒₜₐₗ of the absolute values of the scores at the respective time points. However, the determination method is not limited to this. For example, the determination can be performed in response to establishment of a cell mathematical model between an input factor and an output factor for the cells at a plurality of timings at respective time points of the time-series data. In other words, a mathematical model whose scores at any plurality of time points among the scores Sₜ₀, Sₜ₁, Sₜ₂, Sₜ₃, . . . , Sₜₙ, . . . , and S_{tz} at the respective time points fall within the range of the reference value can be selected. The plurality of time points and the reference value can be set as appropriate.

Alternatively, the determination can be performed in response to establishment of a cell mathematical model between an initial input factor into the cells in the culture data and an output factor calculated continuously over time. In other words, in the foregoing description, the score at each time point is determined by using the difference from the initial culture data, namely, ΔY_{C1,tn}=F(Xₜₙ₊₁, C₁)−F(Xₜ₀, C₁), whereas the production amount of the antibody Y at the time point tn is determined by using ΔY_{C1,tn}=F(Xₜₙ₊₁, C₁)−F(Xₜₙ, C₁). Likewise, the actual measurement value ΔYₜₙ is determined by dividing the amount of antibody production between the time point tn and the time point t0 by a time period tn (ΔYₜₙ=(Yₜₙ₊₁−Yₜ₀)/(tn)). The score Sₜₙ is determined from a difference between an actual measurement value and a calculation value. The scores Sₜ₁, Sₜ₂, Sₜ₃, . . . , Sₜₙ, . . . , and S_{tz} at the respective time points are determined, and a mathematical model whose scores at any plurality of time points fall within the range of the reference value can be selected. The plurality of time points and the reference value can be set as appropriate. Alternatively, a score can be obtained from the sum of the absolute values of the respective scores, and the determination can be performed. The determination may be performed on the basis of both the scores calculated continuously over time and the scores in respective elapsed amounts of time.

If the score falls within the range of the reference value (YES in step S50), it is determined whether to perform determination using a cell simulator (step S52). If the score does not fall within the range of the reference value (NO in step S50), the process returns to the mathematical model creation step (step S46), and a mathematical model is created again. The mathematical model creation step and the mathematical model determination step are repeatedly performed to select a cell mathematical model whose score falls within the range of the reference value.

If the determination using a cell simulator is not to be performed in step S52 (if NO is determined), the determination of the cell mathematical model is terminated, and the process proceeds to the output step (step S58). If the determination using a cell simulator is to be performed (if YES is determined), a determination step using a cell simulator (step S54) is performed.

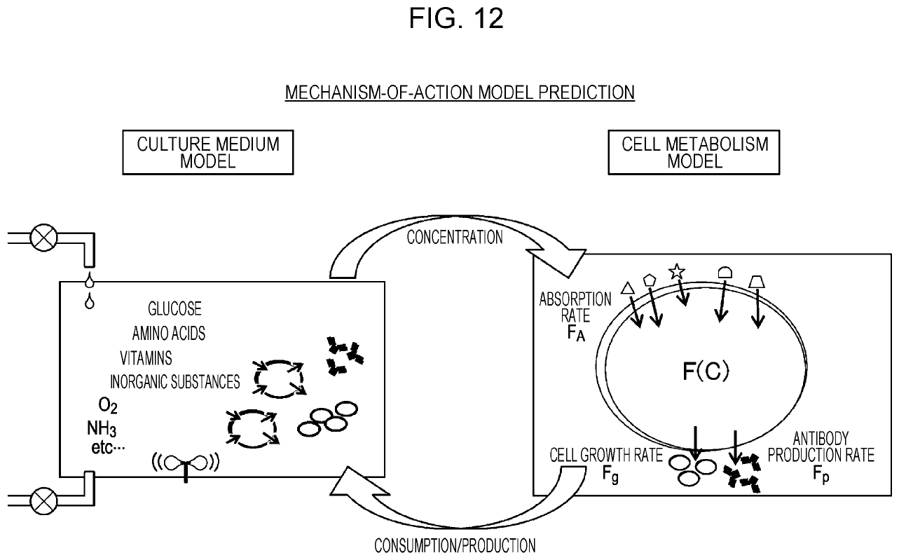

The determination step using a cell simulator (step S54) is performed by calculation using the cell mathematical model created in the mathematical model creation step as an intracellular cell metabolism model. FIG. 12 is a diagram illustrating an overview of a simulation using a cell simulator.

As illustrated in FIG. 12, the calculation using a cell simulator is performed by using respective mathematical models for the inside and the outside of a cell. For the inside of the cell, the cell mathematical model created in the mathematical model creation step is used. For the outside of the cell, a culture medium model for calculating a change in the concentration of each component of the culture medium is used.

For the inside of the cell, the amount of cell growth or antibody production can be determined by calculation in consideration of the theoretical upper limit that can be absorbed by the cell and the consumption rate of the culture medium as described above. For the outside of the cell, the calculation is performed by using a culture medium model. The culture medium model is a model for determining a change in the concentration of a culture medium surrounding the cell when the determination is performed by using the created cell metabolism model, and a cell signaling model represented by a differential equation can be used. Regarding the concentration change, the Runge-Kutta method can be used to solve an ordinary differential equation for the time t to determine the change in the concentration of each component.

A calculation value calculated using the cell simulator is compared with the culture data of the cells input in the culture data input step to check whether the cell mathematical model created in the mathematical model creation step reflects the culture data. At this time, if the calculation value calculated using the cell simulator and the actual culture data are not within the range of the reference value (NO in step S56), the process returns to the mathematical model creation step (step S46), and a cell mathematical model is created. The mathematical model creation step, the mathematical model determination step, and the determination step using a cell simulator are repeatedly performed to select a cell mathematical model for which the calculation value calculated using the cell simulator and the culture data are within the range of the reference value.

If the calculation value calculated using the cell simulator and the actual culture data are within the range of the reference value (YES in step S56), the determination of the cell mathematical model is terminated, and the process proceeds to the output step (step S58).

### Output Step (Step S58)

The output unit 120 of the model determination apparatus performs an output step (step S58). The output step is a step of outputting a cell mathematical model for which the difference from the culture data is determined to fall within the range of the reference value in the mathematical model determination step (step S48) or the determination step using a cell simulator (step S54).

After a cell mathematical model is created, the determination step is performed in a similar manner. Thus, it is checked whether the created cell mathematical model reproduces the input culture data, and a cell mathematical model checked to reproduce the input culture data is output (output step).

The output step is similar to the output step in the method for creating a cell mathematical model, and thus the description thereof will be omitted.

According to the method for determining a cell mathematical model, the cell mathematical model determination program, and the cell mathematical model determination apparatus according to the embodiment of the present invention, created cell mathematical models are determined, thereby making it possible to select a mathematical model that better reproduces culture data from among the created cell mathematical models. As a result, when the mathematical model is used for the prediction of a culture result, the culture result can be predicted with high accuracy. In other words, according to the method for determining a cell mathematical model, the cell mathematical model determination program, and the cell mathematical model determination apparatus according to the embodiment of the present invention, it is possible to evaluate cell characteristics reflecting cellular individuality by using culture data that can be easily acquired without obtaining measurement data that requires a large amount of cost, such as gene expression data.

Cells used to Create Cell Mathematical Model

The cells used in the method for creating a cell mathematical model, the cell mathematical model creation program, the cell mathematical model creation apparatus, the method for determining a cell mathematical model, the cell mathematical model determination program, and the cell mathematical model determination apparatus according to the present embodiment are not specifically limited, and any of eukaryotic cells and prokaryotic cells can be used. Examples of the eukaryotic cells that can be used include animal, plant, or insect derived cell lines, primary cultures, and fungi. Examples of the prokaryotic cells that can be used include bacteria, including Escherichia coli, Bacillus subtilis, cyanobacteria, and actinomycetes, and archaebacteria, including methanogens, hyperhalophiles, and hyperthermophiles.

### Example 1

The following provides more specific description with reference to EXAMPLE.

A simulation was performed to reproduce culture of CHO cell lines in a computer.

As the metabolic circuit information and models of CHO cells, FBA models (iCHOv1_final.xml) were obtained from BiGG Models (http://bigg.ucsd.edu/). Since glucose and 20 types of amino acids are used as the culture medium composition, culture data of glucose and 20 types of amino acids for the CHO cell lines was acquired from Appl Microbiol Biotechnol (2015) 99:4645-4657.

An experiment in which CHO cells producing antibodies were cultured was conducted to acquire time-series culture data. The CHO cell lines were cultured for 14 days, of which from day 2 to day 13, HyClone Cell Boost 7a/b (cytiva) was added once daily to perform culture. The culture data was obtained by measuring the amounts of glucose and 20 types of amino acids, the number of cells, the cell viability, the amount of generated antibodies, the amount of ammonia, and the amount of lactic acid at each of time-series measurement points on days 0, 3, 5, 7, 10, 12, and 14 for the 14 days of culture.

At time points other than 14 days, the culture medium component amounts on a measurement date, and the amounts of change and the rates of change in the culture data between the measurement date and the subsequent measurement date were calculated. An FBA model satisfying the relationship between inputs and outputs was searched for, where the inputs are the culture medium component amounts and the outputs are the amounts of change in the culture data. The search for the FBA model was performed by generating a plurality of virtual FBA models, which are obtained by randomly modifying metabolic circuits of the FBA model (iCHOv1_final.xml), and by using a genetic algorithm. During the search by using the genetic algorithm, calculation values and actual measurement values of a production rate of a cell-produced substance and a cell growth rate, which were calculated by using a virtual FBA model with a culture medium component amount as an input, were determined as score values, and an FBA model having the smallest total sum of the score values for all the time-series points was selected.

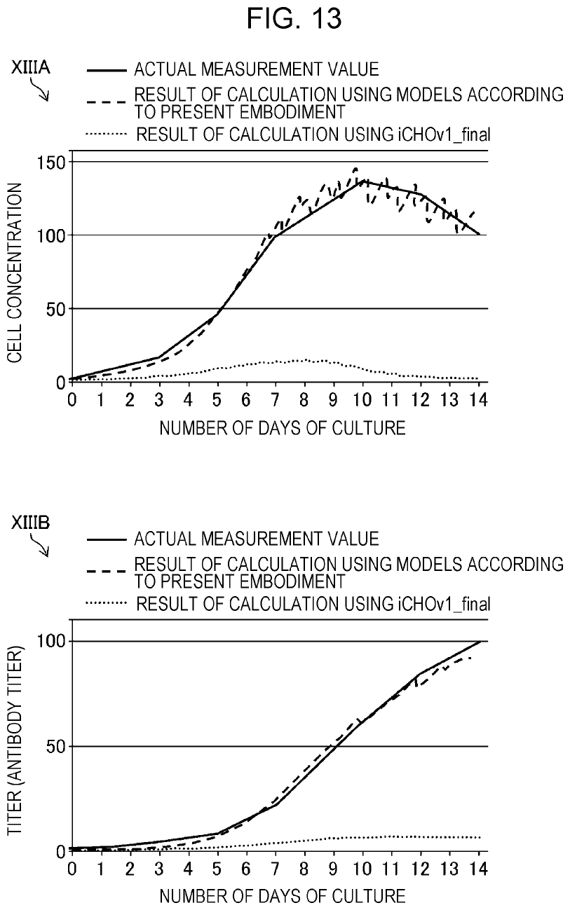

The FBA model acquired from BiGG Models and the estimated FBA model were used to perform dynamic flux balance analysis (dynamic FBA). The results are illustrated in FIG. 13. In FIG. 13, a graph XIIIA is a graph illustrating a relationship between the number of days of culture and the cell concentration, and a graph XIIIB is a graph illustrating a relationship between the number of days of culture and the titer (antibody titer). On the Y axis of the graphs XIIIA and XIIIB, the measurement value of the cell concentration and the measurement value of the titer (antibody titer) on day 14 of culture are set to 100. As illustrated in FIG. 13, it can be found that the culture results calculated by using the FBA model acquired from BiGG Model show a large difference between the actual measurement values and the calculation values and that the reproducibility of culture is poor. In contrast, the culture results calculated by using the estimated FBA model in the present example show a small difference between the actual measurement values and the calculation values, and simulation results that well reproduce the actually measured culture data were obtained.

### REFERENCE SIGNS LIST

10 cell mathematical model creation apparatus
100 processing unit
105 culture data input unit
110 feature value extraction unit
115 mathematical model creation unit
120 output unit
125 display control unit
130 CPU
135 ROM
140 RAM
200 storage unit
300 display unit
310 monitor
400 operation unit
410 keyboard
420 mouse
500 external server
510 external data
600 processing unit
645 mathematical model determination unit
NW network

What is claimed is:

1. A method for creating a cell mathematical model, comprising:
receiving culture data of cells;
extracting feature values of cell activity from the culture data;
creating a cell mathematical model from the feature values; and
outputting the cell mathematical model.

2. The method for creating a cell mathematical model according to claim 1, wherein
the culture data includes the number of cells, an amount of a cell-secreted substance, an amount of a cell-produced substance, an amount of a cell metabolite, a culture medium component amount, temporal change data of the number of cells, temporal change data of the amount of the cell-secreted substance, temporal change data of the amount of the cell-produced substance, temporal change data of the amount of the cell metabolite, and temporal change data of the culture medium component amount.

3. The method for creating a cell mathematical model according to claim 1, further comprising separating the feature values of the cell activity into an input factor into the cells and an output factor from the cells, wherein
in the creating of a cell mathematical model, a cell mathematical model satisfying the input factor and the output factor at any time is created.

4. The method for creating a cell mathematical model according to claim 3, wherein
the feature values of the cell activity are a concentration of a culture medium component, a consumption amount or consumption rate of the culture medium component, a generation amount or generation rate of a cell-secreted substance, a production amount or production rate of a cell-produced substance, and a generation amount or generation rate of a cell metabolite.

5. The method for creating a cell mathematical model according to claim 4, wherein
the input factor is the concentration of the culture medium component, and
the output factor includes the consumption amount or consumption rate of the culture medium component and at least one of the generation amount or generation rate of the cell-secreted substance, the production amount or production rate of the cell-produced substance, or the generation amount or generation rate of the cell metabolite.

6. The method for creating a cell mathematical model according to claim 5, wherein
for the concentration of the culture medium component, a theoretical upper limit amount absorbable by the cells, or a consumption rate is calculated.

7. The method for creating a cell mathematical model according to claim 6, wherein
the theoretical upper limit amount absorbable by the cells, or the consumption rate is calculated by using Michaelis-Menten equation or Fick's law.

8. The method for creating a cell mathematical model according to claim 5, wherein
in the creating of a cell mathematical model, a reference cell mathematical model is randomly modified to generate a cell mathematical model.

9. The method for creating a cell mathematical model according to claim 8, wherein
the reference cell mathematical model is randomly modified by using a genetic algorithm.

10. The method for creating a cell mathematical model according to claim 1, wherein
the cells are eukaryotic cells or prokaryotic cells.

11. The method for creating a cell mathematical model according to claim 10, wherein
the eukaryotic cells are animal, plant, or insect derived cell lines, primary cultures, or fungi.

12. The method for creating a cell mathematical model according to claim 10, wherein
the prokaryotic cells are bacteria including Escherichia coli, Bacillus subtilis, cyanobacteria, or actinomycetes, and archaebacteria including methanogens, hyperhalophiles, or hyperthermophiles.

13. A non-transitory, computer-readable tangible recording medium which records thereon a cell mathematical model creation program for causing, when read by a computer, the computer to execute the method for creating a cell mathematical model according to claim 1.

14. A cell mathematical model creation apparatus comprising a processor configured to:
receive culture data;
extract feature values of cell activity from the culture data;
create a cell mathematical model from the feature values; and
output the cell mathematical model.

15. A method for determining a cell mathematical model, the cell mathematical model being created by the method for creating a cell mathematical model according to claim 1, the method for determining a cell mathematical model comprising:
determining that estimated culture data calculated using the cell mathematical model reflects the culture data.

16. The method for determining a cell mathematical model according to claim 15, wherein
in the determining that the estimated culture data reflects the culture data, it is checked that the cell mathematical model is established between satisfies an input factor and an output factor for the cells at a plurality of timings in time-series data obtained by acquiring the culture data.

17. The method for determining a cell mathematical model according to claim 15, wherein
in the determining that the estimated culture data reflects the culture data, it is checked that the cell mathematical model is established between an initial input factor into the cells in the culture data and an output factor calculated continuously over time.

18. The method for determining a cell mathematical model according to claim 15, wherein
in the determining that the estimated culture data reflects the culture data, a determination is performed using a sum of absolute values of differences between the culture data and the estimated culture data in respective elapsed amounts of time.

19. The method for determining a cell mathematical model according to claim 15, wherein
in the determining that the estimated culture data reflects the culture data, a determination is performed on the basis of a difference between a calculation value obtained by a cell simulator in which the cell mathematical model is incorporated and the culture data.

20. A non-transitory, computer-readable tangible recording medium which records thereon a cell mathematical model determination program for causing, when read by a computer, the computer to execute the method for determining a cell mathematical model according to claim 15.

21. A cell mathematical model determination apparatus comprising a processor configured to:
receive culture data;
extract feature values of cell activity from the culture data;
create a cell mathematical model from the feature values;
output the cell mathematical model; and
determine that estimated culture data calculated using the cell mathematical model reflects the culture data.
